(19) United States
(12) Patent Application Publication
(10) Pub. No.: US 2009/0048632 A1
(43) Pub. Date: Feb. 19, 2009

(54) Spinal Support Rod Kit

(76) Inventors: Paul Firkins, Neuchatel (CH); Marc Sanders, Hengelo (NL); Richard Smith, Memphis, TN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/090,818

(22) PCT Filed: Oct. 20, 2006
(86) PCT No.: PCT/GB2006/003912

§ 371 Date: Aug. 28, 2008

(30) Foreign Application Priority Data
Oct. 22, 2005 (GB) 0521585.0

(51) Int. Cl. A61B 17/70; A61B 17/04

(52) U.S. Cl. 606/246; 606/264; 606/301

(57) ABSTRACT

A spinal support rod kit for the treatment of spinal column shape deformations. The kit comprises a spinal support rod having first and second ends, in which the cross-sectional shape of the rod along at least part of its length, taken perpendicular to its longitudinal axis, includes a first flat side and a rounded side. The kit further comprises an attachment assembly for securing the support rod to the spinal column at least two points along its length. The attachment assembly comprises retaining portions each of which defines a channel for receiving the rod and anchor portions for attachment to a patient's vertebrae. The cross-sectional shape of the channel when viewed from one side includes a first flat side for flush contact with the first flat side of the support rod and a rounded side for tightly engaging the rounded side of the rod so as to restrict movement of the rod through the channel in a direction generally parallel to the axis of the rod.

The present invention relates to a spinal support rod kit for the treatment of spinal column shape deformations.

A spinal support rod can be used in the treatment of spinal column shape deformations. For example, spinal support rods have been used for many years in the treatment of scoliosis. In particular, spinal support rods can be used to treat lateral curvature and/or axial turning of the spinal column. To treat deformations, the spinal support rod is attached to the vertebrae along at least part of the length of the spinal column by an attachment assembly.

It is known to form spinal support rods from shape memory alloy materials, for example as disclosed in EP-A-470660. A rod can be made from a shape memory alloys in a configuration which is stable while the alloy is in its austenite phase, cooled so that the alloy exists in its martensite phase, deformed to a configuration which matches that of the deformed spinal column, attached to the patient's vertebrae, and then heated to a temperature at which the phase of the alloy changes from martensite to austenite. The shape of the rod then tends towards the initial stable configuration, imposing corrective forces on the spine to which it is attached. The viscoelastic qualities of the spinal column allow deformation of the spinal column to be corrected.

It is generally desirable to restrict relative movement between the spinal support rod and the attachment assembly. In particular, it can be important to restrict the rod rotation of the rod within the attachment assembly. It can also be desirable to restrict axial movement of the spinal support rod relative to the attachment assembly. It can further be desirable to allow easy detachment of the spinal support rod from the attachment assembly to allow repositioning of the spinal support rod.

The present invention provides a spinal support rod kit which includes a spinal support rod having a round face and a flat face.

Accordingly, in one aspect, the invention provides a spinal support rod kit for the treatment of spinal column shape deformations, comprising: a spinal support rod having first and second ends, in which the cross-sectional shape of the rod along at least part of its length, taken perpendicular to its longitudinal axis, includes a first flat side and a rounded side, and an attachment assembly for securing the support rod to the spinal column at least two points along its length, comprising retaining portions each of which defines a channel for receiving the rod and anchor portions for attachment to a patient's vertebrae, in which the cross-sectional shape of the channel when viewed from one side includes a first flat side for flush contact with the first flat side of the support rod and a rounded side for tightly engaging the rounded side of the rod so as to restrict movement of the rod through the channel in a direction generally parallel to the axis of the rod.

The provision of a flat side on the rod for flush contact with a flat sides of the channel of the retaining portions helps to restrict the rotation of the rod within the attachment assembly. This is advantageous over known round spinal support rods which can tend to rotate within their attachment assembly.

Further, it has been found that engagement between the rounded side of the rod and the rounded side of the channel can provide sufficient resistance to slippage of the rod through the attachment assembly, while still allowing the spinal support rod to be easily detached from the attachment assembly. This can be in contrast to square rods which prevent slippage of the rod through the attachment assembly by relying on an interference fit between the spinal support rod and the attachment assembly. Such interference fits can be difficult to release which can cause problems when the surgeon needs to revise the position of the spinal support rod.

The provision of a round side to the spinal support rod provides the advantage that the spinal support rod can be less likely to become jammed in the channel of the attachment assembly compared with support rods with a square cross-section.

The control over the engagement of the rod of the present invention in the attachment assembly has the further advantage that it can facilitate precise and accurate location of the support rod in the attachment assembly because of the greater ease with which the rod can be located in the assembly, and then released and relocated as required.

Accordingly, it has been found that the provision of a spinal support rod having a flat side and a rounded side, and an attachment assembly comprising retaining portions each of which defines a channel for receiving the rod in which the cross-sectional shape of the channel includes a rounded side for tightly engaging the rounded side of the rod, can be particularly advantageous as it provides good resistance to axial rotation of the rod within the attachment assembly, allows for easy repositioning of the spinal support rod, while still providing sufficient resistance to axial slippage of the spinal support rod.

The rounded side of the support rod can be regular or irregular in shape when the rod is viewed in cross-section. For example, the rounded side of the support rod can have the approximate shape of a part of an ellipse when the rod is viewed in cross-section. Preferably, the rounded side of the support rod has the approximate shape of an arc of a circle when the rod is viewed in cross-section.

Preferably, the angle subtended by the arc can be selected according to the nature of the engagement that is required between the rod and the channel. Engagement around a greater angle of arc can be preferred in order to provide good resistance to rotation around the axis of the rod, and resistance to axial movement. For example the angle subtended by the arc might be at least about 45°, preferably at least about 60°, possibly at least about 90°, more preferably at least about 120°, especially at least about 150°. It can be particularly preferred that the arc subtends an angle of at least about 170°, for example, about 180°(i.e. the rounded side of the support rod has the approximate shape of a semi-circle when the rod is viewed in cross-section). Smaller angles of arc can be suitable for some applications.

The rounded side of the channel can be regular or irregular in shape. For example, the rounded side of the channel can have the approximate shape of a part of an ellipse when the rod is viewed in cross-section. Preferably, the rounded side of the channel has the approximate shape of an arc of a circle when the channel is viewed from one side.

Preferably, the rounded side of the channel has the approximate shape of an arc of a circle when the channel is viewed from one side. Preferably, the rod is a snug fit in the channel, more preferably, an interference fit so that play between the rod and the channel is minimised, at least when the rod is tightened into the channel. It can be particularly preferred that the radius of the rounded side of the channel is not more than the radius of the rounded side of the support rod. This can help to ensure that the rounded side of the channel tightly engages the rounded side of the support rod to restrict movement of the rod through the channel in a direction generally parallel to the axis of the rod.

It can be preferable that the radius of the rounded side of the channel and the radius of the rounded side of the support rod are such that the channel engages the rod sufficiently to restrict movement of the rod through the channel in a direction generally parallel to the axis of the rod while allowing the surgeon to easily disengage the spinal support rod from the retaining portion of the attachment assembly. Preferably, the rod is larger than the width of the channel so that the internal walls of the channel grip the rod when it is inserted into the channel. Preferably, the difference between the radius of the rounded side of the rod and the radius of the rounded side of the channel is at least about 50 μm, for example about 75 μm. When the rod is larger than the channel in which it is intended to fit, it can be preferred to use an internally threaded circumferential nut which engages a thread on the external wall of the channel in order to retain the rod in the channel. Such a nut can support the walls of the channel against being splayed by the action of the rod on the internal walls of the channel.

When an externally threaded screw is located within the channel, engaging threads on the internal walls of the channel, to retain the support rod in the channel, it will generally preferable for the support rod to be slightly smaller than the width of the channel so that it is a sliding fit within the channel.

Preferably, the cross-sectional shape of the support rod includes a second flat side. For example, the support rod can have two flat sides extending from respective edges of the rounded side. Further, the channel can comprise two flat sides extending from respective of its rounded side, for flush contact with the flat sides of the support rod. In this case, the two flat sides on each of the support rod and the channel can provide resistance to rotation of the spinal support rod within the retaining portion.

Preferably, the cross-sectional shape of the spinal support rod includes second and third flat sides, together with the said first flat side and the said rounded side. Preferably, the cross-sectional shape of the channel also includes second and third flat sides for flush contact with the two flat sides of the spinal support rod, together with the first flat side and the said rounded side. Accordingly, all three flat sides can both provide resistance to the rotation of the spinal support rod within the retaining portion.

Preferably, the rod which includes three flat sides is arranged so that two of the flat sides are extend approximately parallel to one another, with the rounded side and the third flat side located opposite to one another, along opposite edges of the parallel sides.

The first flat side of the support rod can be opposite to the rounded side. For example, the support can comprise the first flat side and the rounded side only. In this case, only the first flat side provides resistance to rotation of the spinal support rod relative to the attachment assembly. When the rounded side has the shape of a portion of the circumference of a circle (in other words, is an arc), it can be preferred for the ratio of the radius of that circle to the width of the opposite flat side to be at least about 0.6, more preferably at least about 0.7, for example at least about 0.8. The value of the ratio will generally be not more than about 1.5.

Preferably, the first flat side of the support rod is adjacent to the rounded side. It has been found that great resistance to the rotation of the spinal support rod within the retaining assembly can be provided when the first flat side of the support rod is adjacent to the rounded side.

It can be advantageous to provide for significant resistance to rotation of the spinal support rod within the attachment assembly at the ends of the spinal support rod. Preferably, the side of the support rod that is rounded along at least part of its length is flat at least one end of the rod. For example, preferably the cross-sectional shape of the support rod towards at least one end of the rod is approximately square. Preferably, the rounded side of the channel of the retaining portion of the attachment assembly that secures one of the ends of the spinal support rod to the spinal column is flat. For example, preferably the cross-sectional shape of the channels of the retaining portions of the attachment assembly that secure the ends of the spinal support rod to the spinal column is approximately square. Preferably, the rounded sides of the channels of the retaining portions of the attachment assembly that secures the spinal support rod to the spinal column towards the ends of the spinal support rod, are flat.

As discussed in the paragraph below, it can be advantageous to allow for some movement of the spinal support rod within the channel of a retaining portion at points in-between the ends of the spinal support rod, and particularly towards the middle of the spinal support rod. Accordingly, in some circumstances it can be preferable for the cross-sectional shape of the spinal support rod to be rounded towards its middle so that the force by which the spinal support rod is held against rotation and sliding within the channel of a retaining portion towards the middle of the spinal support rod is less than that at the ends of the spinal support rod.

Preferably, the kit includes first and second retaining portions for securing first and second ends of the spinal support rod to the spinal column at spaced apart points along the spinal column. Preferably, the kit includes a third retaining portion for securing the spinal support rod to the spinal column at a point between the first and second ends of the support rod. In some circumstances it can be preferable that the forces by which the rod is held against rotation and sliding within the channels of the first and second retaining portions is greater than the forces by which the rod is held against rotation and sliding within the channel of the third retaining portion. In this way, the support rod will be restrained against movement relative to attachment assemblies at its ends, but limited movement can be permitted at points along its length. Such limited movement can be desirable for some patients. It can be achieved by restricting the tension that can be applied to a spinal support rod within a fixation channel. For example, when the rod is retained within a channel by means of a threaded fastener such as a screw or a nut or both, one of the threads on the channel and the fastener can include a discontinuity to restrict relative rotation between the channel and the fastener.

The cross-sectional shape of the spinal support rod can be approximately constant along at least part, especially the central part, of its length.

The cross-sectional area of the support rod can be approximately constant along its length. It can be advantageous for the cross-sectional area of the spinal support rod to be greater at some points along its length than at other points. It can be preferable that the rod cross-sectional area of the spinal support rod is greater at points which will be subject to greater forces. Preferably, the cross-sectional area of the support rod is greater at least one of its ends than at its other end. More preferably, the cross-sectional area of the spinal support rod is greater at its end that will be proximal the lumbar area of the spinal column.

The shape and size of the spinal support rod and the channel of the retaining portion can be configured so that the spinal support rod can be secured within the channel by way of an interference fit between the spinal support rod and the retaining device.

The channel can be open so that the spinal support rod can be slid into the channel in a direction generally transverse to the axis of the spinal support rod.

Preferably, the kit includes a clamping device for clamping the spinal support rod within the channel. Examples of suitable clamping devices include those which are used in existing spinal support assemblies. For example, the Moss Miami system which is sold by DePuy Spine Inc includes circumferentially threaded screws which can engage a thread on the internal wall of a rod-receiving channel, and includes internally threaded nuts which can engage a thread on the external wall of a rod-receiving channel. Such screws or such nuts or both can be used in the kit of the present invention.

Preferably, the clamping device can be separated from and subsequently reattached to the channel. This can be advantageous as the clamping device can be removed to allow for the spinal support rod to be slid into and out of the channel in a direction generally transverse to the axis of the spinal support rod.

The clamping device can provide the first flat side of the channel. The clamping device can provide additional flat sides of the channel. Optionally, the clamping device can provide the rounded side of the channel.

A wall of the channel can be threaded. The thread can be provide on an external face of the channel. The thread can be provided on an internal wall of the channel. Preferably, the clamping device comprises a screw which can engage the thread on the channel, so that it can be tightened to clamp the spinal support rod between the screw and the rounded side of the channel.

The anchor portion can be any type of mechanism by which the retaining portion can be attached to a patient's vertebrae. Preferred mechanisms include those disclosed in EP-A-470660 and U.S. Pat. No. 5,391,168.

The spinal support rod will generally be formed from a metal. Suitable materials can include titanium and its alloys, and certain stainless steels. Preferably, the spinal support rod is formed from a shape memory alloy. For example, the spinal support rod can be made from nickel titanium based shape memory alloy. Other materials for the rod might include titanium and its alloys. Fittings for use with the support rod, such as connectors between rod segments and devices for fixing the rod to a patient's vertebrae can be made from titanium and its alloys, or from stainless steel. The selection of materials for spinal support rods and for fittings for use therewith can be in accordance with existing products from DePuy Spine Inc and from other companies.

The spinal support rod can be made using conventional techniques for making rods, with appropriate modification for making the spinal support rod according to the invention. Techniques including milling, grinding, and hot and cold working techniques such as drawing. The rod can initially be made as a round rod using conventional techniques and then modified to include a flat side by techniques such as milling or grinding.

In another aspect, the invention provides a spinal support rod for use in the treatment of spinal column shape deformations, in which the cross-sectional shape of the spinal support rod taken perpendicular to its length, comprises a first flat side and a rounded side.

Embodiments of the invention will now be described, by way of example only, and with reference to the accompanying drawings, in which:

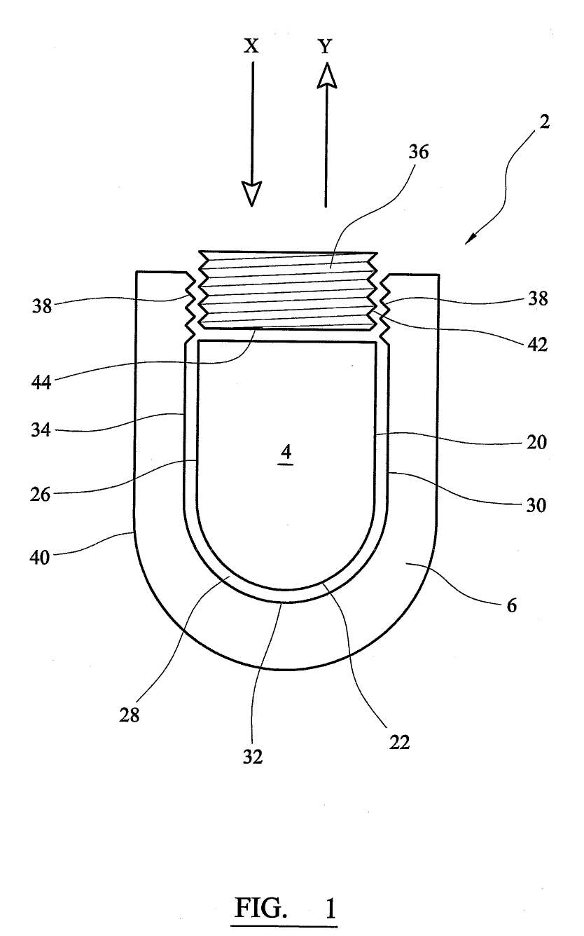

FIG. 1 is an end view of a spinal support rod received within a retaining portion of a spinal support rod kit according to the invention; and

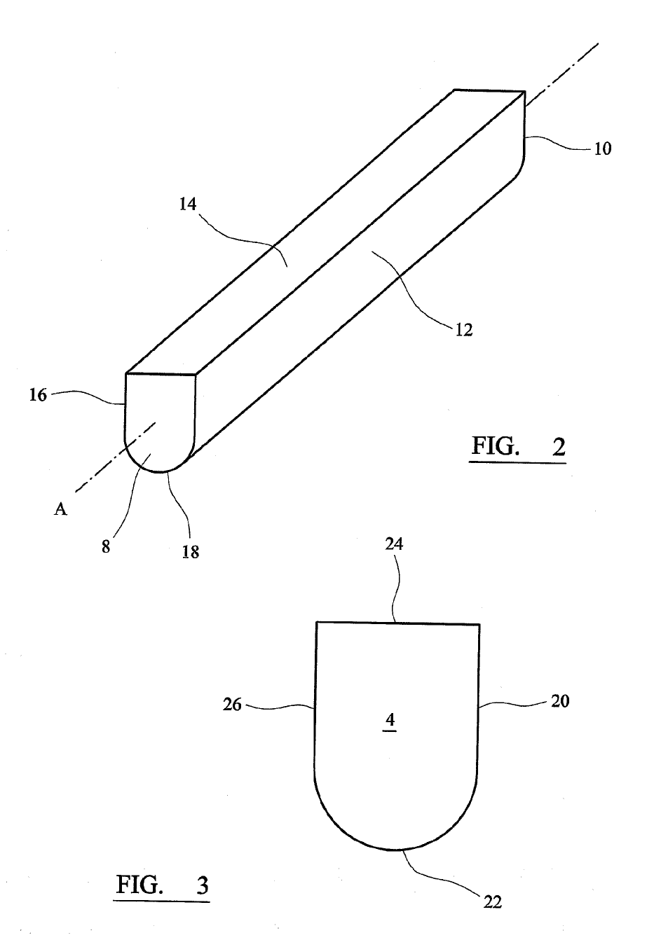

FIG. 2 is a perspective view of the spinal support rod shown in FIG. 1; and

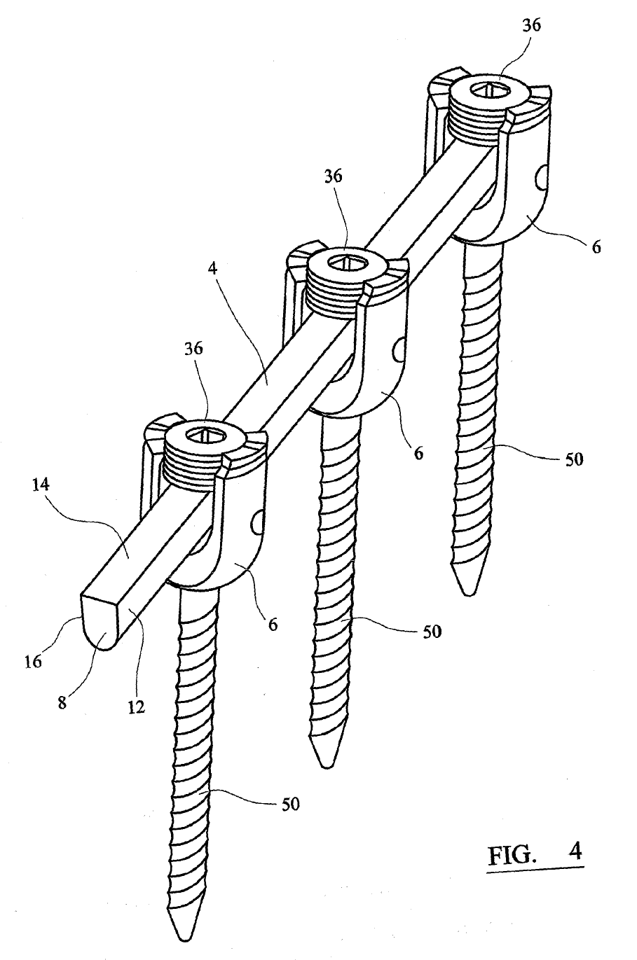

FIG. 3 is a cross-sectional view of the spinal support rod shown in FIG. 1, taken perpendicular to its longitudinal axis.

FIG. 4 is an isometric view of a spinal support rod and fixation screws which can be used to fix it to a patient's vertebrae.

Referring to the drawings, FIG. 1 shows a spinal support rod kit 2 which comprises a spinal support rod 4 and a retaining portion 6 of an attachment assembly. The spinal support rod kit 2 is shown in isolation for simplicity and ease of illustration. However, it will be appreciated that the retaining portion 6 will be attached to a vertebra by an anchor portion (not shown). The anchor portion can be any suitable mechanism for securing the retaining portion 6 to a vertebra, such as a pedicle screw, or a hook. It will also be appreciated that the attachment assembly of the spinal support rod kit will comprise a plurality of retaining portions 6. Typically, at least two retaining portions 6 will be provided for securing the spinal support rod 4 at two points to the spinal column, across the part of the spinal column that is to be treated. FIG. 4 shows the spinal support rod 4 received within three retaining portions 6. In this embodiment, the spinal support rod 4 will be secured to a patient's spinal column at three points. As illustrated, the retaining portions have fixation screws 50 for fixing the retaining portions to a patient's vertebrae (not shown).

With reference to FIG. 2, the spinal support rod 4 has first 8 and second 10 ends. First 12, second 14 and third 16 flat walls and a rounded wall 18 extend between the first 8 and second 10 ends of the spinal support rod 4.

As shown in FIG. 3, the cross-sectional shape of the spinal support rod 4, taken in a plane perpendicular to its longitudinal axis A, comprises a first flat side 20 defined by the first flat wall 12, and a rounded side 22 defined by the rounded wall 18. The cross-sectional shape further includes second 24 and third 26 flat sides defined by second 14 and third 16 flat walls. The cross-sectional shape and size of the spinal support rod 4 is approximately constant along its entire length between the first 8 and second 10 ends.

Referring back to FIG. 1, the wall 40 of the retaining portion 6 is approximately U-shaped when viewed from one side. The retaining portion 6 of the attachment assembly defines a channel 28 for receiving the spinal support rod 4. The cross-sectional shape of the channel 28 includes first 30 and second 34 flat sides for flush contact with the first 20 and second 26 flat side 20 of the spinal support rod 4, and a rounded side 32, extending between the first 30 and second flat 34 sides, which engages the rounded side 22 of the spinal support rod when the spinal support rod is fully received within the retaining portion 6. The spinal support rod 4 and the retaining portion 6 are shaped and sized so that the spinal support rod is a snug fit within the channel 28.

The wall 40 of the retaining portion 6 is threaded at the ends 38 of the first 30 and second 34 flat sides distal to the rounded side 32, for threaded engagement with a clamping device 36 (described in more detail below).

The retaining device 6 comprises a clamping device 36. In the embodiment shown, the clamping device 36 is a set screw having a threaded wall 42 for threaded engagement with the threaded on the wall 40 of the retaining portion 6. The set screw has a first end 46 for engagement with a tool (not shown) to rotate the set screw, and a second end 44. When the set screw is received within the walls 40 of the retaining portion, the second end 44 provides a third flat side of the channel 28 for flush contact with the second flat side 14 of the spinal support rod 4.

In the embodiment shown, the radius of the rounded side 22 of the spinal support rod 4 is approximately 3.0 mm, and the radius of the rounded side 32 of the retaining portion 6 is approximately 3.05 mm Accordingly, the difference between the radii is approximately 0.05 mm. The spinal support rod is therefore a sliding fit in the channel which is defined within the retaining portion. Locating the rod within the channel does not require outward deformation of the walls of the channel.

In use, the retaining portion 6 is secured to a spinal column via an anchor portion (not shown). The clamping device 36 is initially removed from the walls 40 of the retaining portion 6 and the spinal support rod 4 is slid into the channel 28 in a direction perpendicular to the axis of the spinal support rod, as illustrated by arrow X.

When the spinal support rod 4 is fully received within the retaining portion 6, the first 20 and third 26 flat sides of the spinal support rod fit flush against the first 30 and second 34 flat sides of the channel 28, thereby restricting rotation of the spinal support rod within the retaining portion 6.

Also, the difference in the radii of the rounded 22 side of the spinal support rod 4 and the rounded side 32 of the retaining portion 6 is such that when the spinal support rod is fully received in the retaining portion, the rounded side of the retaining portion tightly engages the rounded side of the spinal support rod. This engagement restricts sliding of the spinal support rod 4 through the retaining portion in a direction approximately parallel to its longitudinal axis.

Once the spinal support rod 4 is fully received within the retaining portion 6, the set screw 36 is fastened to the walls 40 of the retaining portion and is tightened so that the spinal support rod 4 is clamped between the third flat side of the channel 28 provided by the flat end 44 of the set screw and the rounded side 32 of the retaining portion.

When the spinal support rod 4 is properly clamped in the retaining portion 6, the spinal support rod is further restricted from sliding through the retaining portion in a direction approximately parallel to its longitudinal axis.

To remove the spinal support rod 4 from the retaining portion 6, the set screw 36 is loosened and removed from the walls 40 of the retaining portion. The spinal support rod 4 is then pulled out of the retaining portion 6 in a direction perpendicular to its longitudinal axis as illustrated by arrow Y.

1. A spinal support rod kit for the treatment of spinal column shape deformations, comprising:
a spinal support rod having first and second ends, in which the cross-sectional shape of the rod along at least part of its length, taken perpendicular to its longitudinal axis, includes a first flat side and a rounded side, and
an attachment assembly for securing the support rod to the spinal column at least two points along its length, comprising retaining portions each of which defines a channel for receiving the rod and anchor portions for attachment to a patient's vertebrae,
in which the cross-sectional shape of the channel when viewed from one side includes a first flat side for flush contact with the first flat side of the support rod and a rounded side for tightly engaging the rounded side of the rod so as to restrict movement of the rod through the channel in a direction generally parallel to the axis of the rod.

2. A spinal support rod kit as claimed in claim 1, in which the rounded side of the support rod has the approximate shape of an arc of a circle when the rod is viewed in cross-section.

3. A spinal support rod kit as claimed in claim 2, in which the arc subtends an angle of at least about 90°.

4. A spinal support rod kit as claimed in claim 1, in which the rounded side of the channel has the approximate shape of an arc of a circle when the channel is viewed from one side.

5. A spinal support rod kit as claimed in claim 2, in which the rounded side of the channel has the approximate shape of an arc of a circle when the channel is viewed from one side, and in which the radius of the rounded side of the channel is not more than the radius of the rounded side of the rod.

6. A spinal support rod kit as claimed in claim 1, in which the first flat side of the support rod is adjacent to the rounded side.

7. A spinal support rod kit as claimed in claim 1, in which the first flat side of the support rod is opposite to the rounded side.

8. A spinal support rod kit as claimed in claim 1, in which the cross-sectional shape of the support rod includes at least one further flat side.

9. A spinal support rod kit as claimed in claim 1, in which the cross-sectional shape of the support rod includes two flat sides, together with the said first flat side and the said rounded side.

10. A spinal support rod kit as claimed in claim 1, in which the side of the support rod that is rounded along at least part of its length is flat at least one end of the rod.

11. A spinal support rod kit as claimed in claim 10, in which the cross-sectional shape of the support rod towards the said end is approximately square.

12. A spinal support rod kit as claimed in claim 1, in which the cross-sectional area of the support rod is greater at one of its ends than at its other end.

13. A spinal support rod kit as claimed in claim 1, in which the retaining device comprises a clamping device for clamping the spinal support rod within the channel, the clamping device providing the first flat side of the channel.

14. A spinal support rod kit as claimed in claim 13, in which a wall of the channel is threaded, and in which the clamping device comprises a screw which can engage the thread on the channel, so that it can be tightened to clamp the spinal support rod between the screw and the rounded side of the channel.

15. A spinal support rod kit as claimed in claim 1, which is formed from a shape memory alloy.

16. A spinal support rod for use in the treatment of spinal column shape deformations, in which the cross-sectional shape of the spinal support rod taken perpendicular to its length, comprises a first flat side and a rounded side.
